(19) United States
(12) Patent Application Publication
(10) Pub. No.: US 2021/0315462 A1
(43) Pub. Date: Oct. 14, 2021

(54) PHYSIOLOGICAL SIGNAL PROCESSING SYSTEM AND PHYSIOLOGICAL SIGNAL PROCESSING METHOD

(71) Applicant: Quanta Computer Inc., Taoyuan City (TW)

(72) Inventors: Yu-Siang WANG, Taoyuan City (TW); Yung-Ming CHUNG, Taoyuan City (TW)

(21) Appl. No.: 17/000,740

(22) Filed: Aug. 24, 2020

(30) Foreign Application Priority Data
Apr. 9, 2020 (TW) 109111890

(51) Int. Cl. A61B 5/0205; A61B 5/0402; A61B 5/11; A61B 5/00; G16H 20/30

(52) U.S. Cl. CPC A61B 5/0205; A61B 5/0402; A61B 5/1118; A61B 5/6823; A61B 5/0245; A61B 5/4561; A61B 5/7225; G16H 20/30; A61B 5/6833

(57) ABSTRACT

A physiological signal processing method includes the following steps: receiving a plurality of ECG signals and user information; capturing the ECG signals; detecting the displacement state of the ECG monitoring device to obtain three-axis data; calculating the heart rate based on an ECG or the ECG signals, and calculating an activity amount based on the three-axis data; and generating an activity intensity based on the activity amount, the user information, and the heart rate.

### CROSS REFERENCE TO RELATED APPLICATIONS

This application claims priority of Taiwan Patent Application No. 109111890, filed on Apr. 9, 2020, the entirety of which is incorporated by reference herein.

### BACKGROUND OF THE INVENTION

### Field of the Invention

The present disclosure relates to a signal processing system and, in particular, to a physiological signal processing system and physiological signal processing method.

### Description of the Related Art

In general, when measuring an electrocardiogram (ECG), it is necessary to perform measurement while the examinee is at rest to obtain accurate data. With the popularization of wearable products, some wearable devices (such as smart watches, bracelets, and ECG chest straps) can also measure physiological signals to generate and display an ECG. The physiological signal is, for example, an ECG signal. When the patient uses the wearable device, the physiological signal measured by the wearable device may be affected by the movement of the body.

After the wearable device generates an ECG, the doctor or nurse will interpret the ECG to observe the physiological state of the examinee. Especially before and after surgery, the patient's ECG needs to be interpreted correctly. If the examinee moves while his physiological signals are being measured, the waveform of the ECG may be abnormal. It is difficult for the doctor or nurse to determine whether the abnormality is caused by the patient's symptoms, or if the abnormality is caused by the examinee's body moving during the ECG.

Therefore, how to allow medical personnel to interpret ECGs more accurately and reduce ECG distortion caused by the posture or activity of the patient during the measurement of ECG signals, which makes it difficult for medical personnel to interpret the ECG, has become a problem in the art that needs to be solved.

### BRIEF SUMMARY OF THE INVENTION

In order to solve the above problems, the present disclosure provides a physiological signal processing system. The physiological signal processing system includes an electrocardiogram (ECG) monitoring device. The ECG monitoring device comprises a processor, an ECG module and a gravity sensor (g-sensor). The processor is configured to receive a plurality of ECG signals and user information. The ECG module is configured to capture the ECG signals and transmit the ECG signals to the processor. In addition, the gravity sensor is configured to detect the displacement state of the ECG monitoring device to obtain three-axis data. In addition, the processor calculates the heart rate based on an ECG or the ECG signals, calculates an activity amount based on the three-axis data, and generates an activity intensity based on the activity amount, the user information, and the heart rate.

In accordance with one feature of the present invention, the present disclosure provides a physiological signal processing method. The physiological signal processing method includes the following steps: receiving a plurality of ECG signals and user information; capturing the ECG signals; detecting the displacement state of the ECG monitoring device to obtain three-axis data; calculating the heart rate based on an ECG or the ECG signals, calculating an activity amount based on the three-axis data; and generating an activity intensity based on the activity amount, the user information, and the heart rate.

The physiological signal processing method and physiological signal processing system shown in the present invention can output information about the posture, the activity amount and the activity intensity of patient's body in combination with the waveform of the ECG, in order to allow the user to know the patient's posture, the activity amount and the activity intensity at each time point of the ECG. Labeling the posture, activity amount and the activity intensity at each time point of the ECG can assist medical personnel to interpret ECGs more accurately, and reduce ECG distortion due to the patient's posture or activity at the time of the ECG measurement, making interpretation difficult for medical personnel.

### BRIEF DESCRIPTION OF THE DRAWINGS

In order to describe the manner in which the above-recited and other advantages and features of the disclosure can be obtained, a more particular description of the principles briefly described above will be rendered with reference to specific examples thereof which are illustrated in the appended drawings. Understanding that these drawings depict only exemplary aspects of the disclosure and are not therefore to be considered to be limiting of its scope, the principles herein are described and explained with additional specificity and detail through the use of the accompanying drawings in which:

FIG. 1A is a schematic diagram of a physiological signal processing system in accordance with one embodiment of the present disclosure.

FIG. 1B is a block diagram of an electrocardiogram (ECG) monitoring device in accordance with one embodiment of the present disclosure.

FIG. 2 is a schematic diagram of a physiological signal processing system in accordance with one embodiment of the present disclosure.

FIG. 3A is a flowchart of a physiological signal processing method in accordance with one embodiment of the present disclosure.

FIG. 3B is a schematic diagram of an ECG 350 in accordance with one embodiment of the present disclosure

FIG. 4 is a flowchart of a body-posture determination method in accordance with one embodiment of the present disclosure.

FIG. 5A is a flowchart of an activity amount calculation method in accordance with one embodiment of the present disclosure.

FIG. 5B is a schematic diagram of X-axis data of baseline drift according to an embodiment of the present invention.

FIG. 5C is a schematic diagram of X-axis data after baseline cancellation process according to an embodiment of the present invention.

FIG. 5D is a schematic diagram of a line chart for calculating the activity amount according to an embodiment of the present invention.

FIG. 6 is a flowchart of an activity intensity generation method in accordance with one embodiment of the present disclosure.

FIG. 7 is a schematic diagram illustrating an activity intensity generation method according to an embodiment of the present invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The present invention will be described with respect to particular embodiments and with reference to certain drawings, but the invention is not limited thereto and is only limited by the claims. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or groups thereof.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

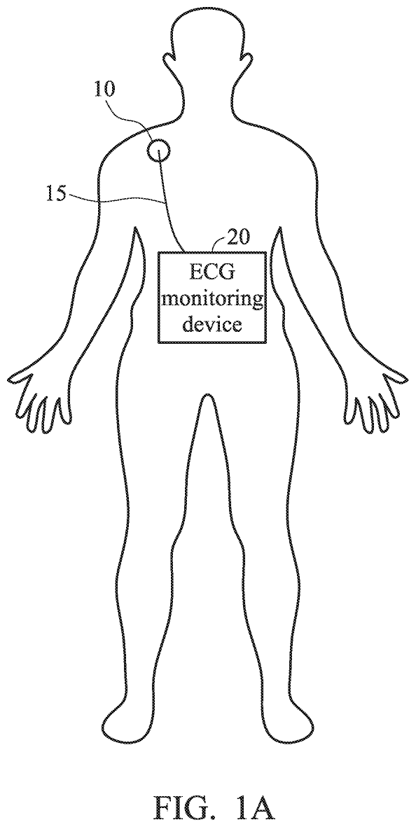

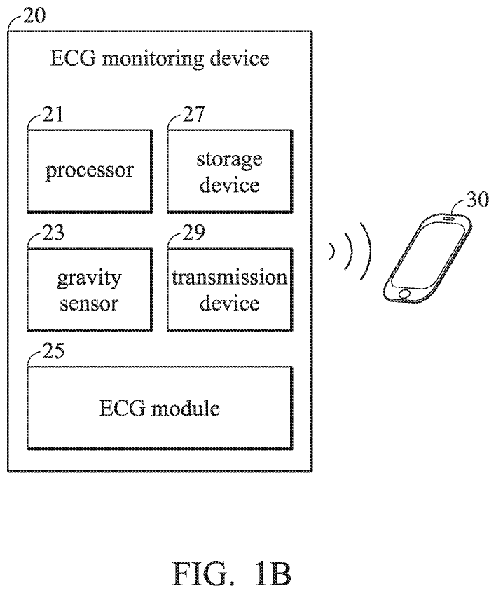

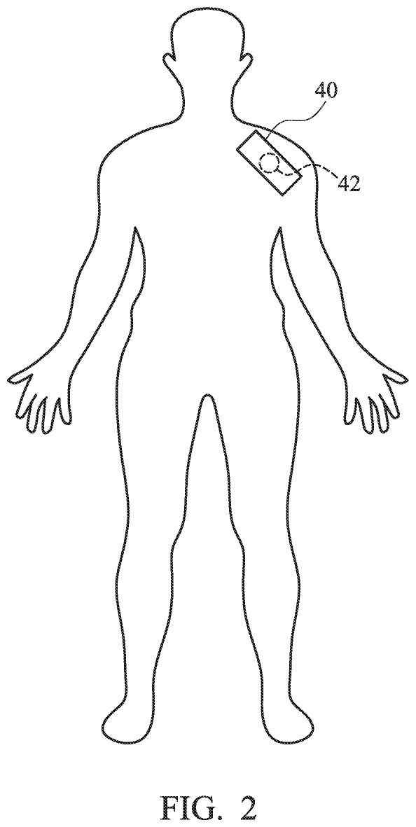

Referring to FIGS. 1A-1B and 2, FIG. 1A is a schematic diagram of a physiological signal processing system in accordance with one embodiment of the present disclosure. FIG. 1B is a block diagram of an electrocardiogram (ECG) monitoring device 20 in accordance with one embodiment of the present disclosure. FIG. 2 is a schematic diagram of a physiological signal processing system in accordance with one embodiment of the present disclosure.

In one embodiment, as shown in FIG. 1A, the physiological signal processing system includes an electrocardiogram (ECG) monitoring device 20. The ECG monitoring device 20 is worn on the human body at a horizontal wearing angle. In one embodiment, the physiological signal processing system further includes an ECG electrode patch 10. The ECG electrode patch 10 is pasted under the right shoulder blade of the human body to detect the ECG signal, and the ECG electrode patch 10 transmits the ECG signal by wire 15 to the ECG monitoring device 20 located below the left chest.

In one embodiment, since the human body itself is a conductor, changes in the electrical potential of the conductive tissue around the heart will produce a weak current to the body surface. When the weak current flows through the whole body, the ECG electrode patch 10 attached to the body surface is used to receive the weak current generated by the contraction and expansion of the heart. This weak current can be regarded as an ECG signal, and the ECG electrode patch 10 can transmit the received ECG signal to the ECG monitoring device 20.

In one embodiment, the ECG electrode patch 10 can be implemented using a product implemented by the known technology.

In one embodiment, as shown in FIG. 1B, the ECG monitoring device 20 includes a processor 21, an ECG module 25 and a gravity sensor (g-sensor) 23. In one embodiment, the ECG monitoring device 20 further includes a storage device 27 and a transmission device 29.

In one embodiment, the processor 21 can be any device with an arithmetic function. In one embodiment, the processor 21 can be implemented by an integrated circuit such as a micro controller, a microprocessor, a digital signal processor, an application specific integrated circuit (ASIC), or a logic circuit. In one embodiment, the ECG module 25 receives the ECG signal and transmits the ECG signal to the processor 21. In one embodiment, the processor 21 transmits the ECG signal to the storage device 27 to store. In one embodiment, the processor 21 is used to process the ECG signal and send the processed (e.g., aggregated) ECG signal to the storage device 27 or the transmission device 29.

In one embodiment, the ECG module 25 can integrate multiple ECG signals into ripples with high and low fluctuations, and the pattern formed by the ripples is called an ECG. In one embodiment, the ECG electrode patch 10 can be implemented using a product implemented by the known technology.

In one embodiment, the gravity sensor 23 is also called a linear accelerometer. In addition, the gravity sensor 23 can provide information on speed and displacement. In one embodiment, the gravity sensor 23 can be used to measure the tilt angle of the ECG monitoring device 20. In one embodiment, the gravity sensor 23 can be implemented using products implemented by the known technology.

In one embodiment, the storage device 27 can be implemented as a read-only memory, flash memory, floppy disk, hard disk, optical disk, pen drive, tape, database accessible by network, or those skilled in the art can easily think of storage media with the same functions.

In one embodiment, the transmission device 29 can be a Bluetooth transmission device, a Wi-Fi transmission device, or other wired or wireless transmission devices. The transmission device 29 can transmit the ECG signal or other information (such as user information) to the electronic device 30.

In an embodiment, the electronic device 30 is, for example, a mobile phone, a tablet, or other devices with computing functions.

In one embodiment, as shown in FIG. 2, the physiological signal processing system includes an ECG monitoring device 40. The ECG electrode patch 42 for obtaining the ECG signal is affixed to the back side of the ECG monitoring device 40 (the ECG monitoring device 40 can be adhered to the side of the body surface). In addition, the ECG electrode patch 42 is obliquely attached to the left chest of the human body at an oblique wearing angle (for example, 45 degrees). The ECG monitoring device 40 is worn on the human body at an oblique wearing angle. In one embodiment, the internal components of the ECG monitoring device 40 in FIG. 2 are the same as those in FIG. 1B. The ECG monitoring device 40 in FIG. 2 fixes the ECG electrode patch 42 to the rear side of the ECG monitoring device 40 to facilitate adhesion to the human body.

In one embodiment, the internal components of the ECG monitoring device 40 in FIG. 2 are the same as the internal components of the ECG monitoring device 20 in FIG. 1B. The ECG electrode patch 42 in FIG. 2 is electrically coupled with the processor 21 in the ECG monitoring device 40 to integrate the ECG electrode patch 42 with the ECG monitoring device 40. In addition, the ECG electrode patch 42 is affixed to the back side of the ECG monitoring device 40. In one embodiment, after the ECG electrode patch 42 receives the ECG signal, the ECG electrode patch 42 transmits the ECG signal to the ECG module 25.

As can be seen from FIG. 1A, the ECG monitoring device 20 is worn horizontally on the human body. The ECG monitoring device 40 of FIG. 2 is worn on the human body at an oblique wearing angle (for example, 45 degrees). Therefore, the wearing angle of the ECG monitoring device 20 of FIG. 1A and the ECG monitoring device 40 of FIG. 2 are different. In addition, the ECG monitoring device 20 of FIG. 1A and the ECG monitoring device 40 of FIG. 2 are also worn at different positions on the human body.

In addition, the ECG electrode patch 10 of FIG. 1A is electrically coupled to the ECG monitoring device 20 through the wire 15. The ECG electrode patch 42 in FIG. 2 is fixed (or integrated) in the ECG monitoring device 40. For example, the ECG electrode patch 42 is fixed to the back side of the ECG monitoring device 40, so that the ECG electrode patch 42 can be adhered to the surface of the body.

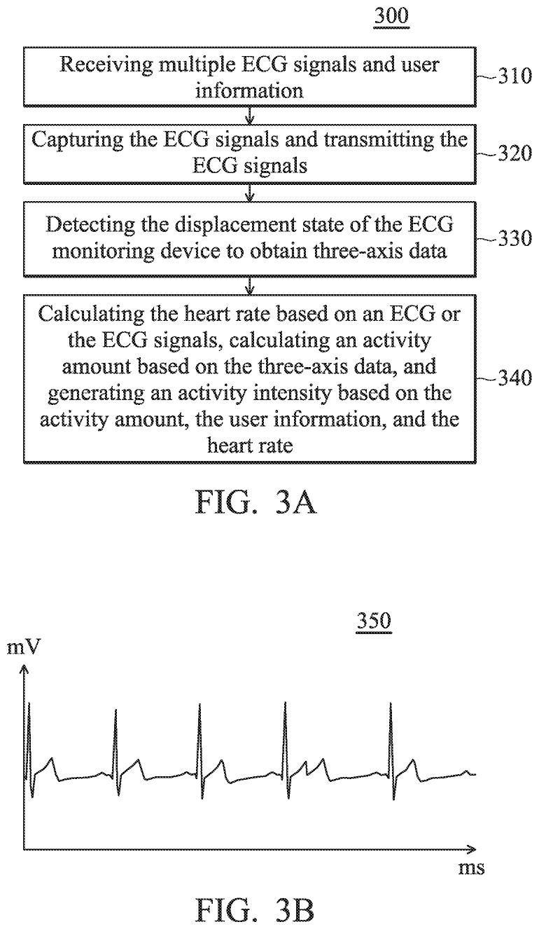

Referring to FIG. 3A, FIG. 3A is a flowchart of a physiological signal processing method 300 in accordance with one embodiment of the present disclosure. The physiological signal processing method 300 can be implemented by using the physiological signal processing system shown in FIG. 1A or FIG. 2. For convenience of description, the following uses the ECG monitoring device 20 and the ECG electrode patch 10 as an example for description.

In step 310, the ECG module 25 is used to receive multiple ECG signals, and the electronic device 30 is used to receive user information. In addition, the user information is transmitted from the electronic device 30 to the processor 21.

In one embodiment, when the ECG electrode patch 10 (shown in FIG. 1A) or the ECG electrode patch 42 (shown in FIG. 2) attached to the surface of the body receives the ECG signal, the ECG signal is sent to the ECG module 25.

In one embodiment, the user information includes the weight of the examinee. In one embodiment, the user information includes information such as the examinee's weight, height, body fat, age, and/or gender. In one embodiment, the user information can be input by the user through an input interface of the electronic device 30, and the electronic device 30 transmits the user information to the processor 21, such as a touch screen and/or entity button. In one embodiment, the processor 21 can store the received user information in the storage device 27.

In step 320, the ECG module 25 captures the ECG signals and transmits the ECG signals to the processor 21. In one embodiment, the ECG module 25 records these ECG signals to generate an ECG.

Please refer to FIG. 3B, which is a schematic diagram of an ECG 350 in accordance with one embodiment of the present disclosure. As shown in FIG. 3B, the processor 21 outputs an ECG 350 after receiving the ECG signals from the ECG module 25. The ECG 350 can be a graph, the horizontal axis (X axis) represents time (unit may be milliseconds, ms), and the vertical axis (Y axis) represents voltage (unit may be millivolts, mV).

However, those skilled in the art should understand that FIG. 3B is only a schematic diagram. The generation of the ECG 350 can be drawn by the ECG module 25 by applying known techniques. For example, the concept of ECG coordinate paper is applied (in general, in the ECG coordinate graph paper, the horizontal axis represents time in milliseconds, and the vertical axis represents amplitude, that is, voltage in millivolts. Each small grid on the horizontal axis is 40 milliseconds; every 5 small grids (1 large grid) are 200 milliseconds. The distance between the two large grids on the vertical axis is calibrated to represent 1 millivolt, and it will not further described herein.

In step 330, the gravity sensor 23 detects the displacement state of the ECG monitoring device 20 to obtain three-axis data.

In one embodiment, the gravity sensor 23 works based on the basic principle of acceleration, which is a space vector. Sensing the components of the ECG monitoring device 20 on three coordinate axes (X-axis, Y-axis, and Z-axis) through the gravity sensor 23, it can be used to estimate the motion state of the ECG monitoring device 20 (the motion state can be regarded as the human posture, for example, lying on the side, lying down, or standing upright).

On the other hand, when the posture of the human body is not known in advance, the gravity sensor 23 can be applied to detect the acceleration signal. Since the gravity sensor 23 is also based on the principle of gravity to work, the gravity sensor 23 will output different voltage values at different tilt angles, and the numerical value will be obtained after the program conversion. Therefore, it can be used to measure the tilt angle of the ECG monitoring device 20. The gravity sensor 23 can measure the three-axis acceleration and tilt angle of the electrocardiogram monitoring device 20 in space, and can reflect the posture of the human body.

In one embodiment, the three-axis data includes the gravity sensor 23 sensing the component of the ECG monitoring device 20 on the three coordinate axes, the three-axis acceleration, and/or the tilt angle. In one embodiment, the gravity sensor 23 transmits the sensed three-axis information to the processor 21. The processor 21 determines the posture of the human body according to the three-axis information, for example, lying on the side, lying down, or standing upright.

For example, the processor 21 can obtain the three-dimensional coordinates of the ECG monitoring device 20 in the space center according to the components on the three coordinate axes (X axis, Y axis, and Z axis). The processor 21 adds the squares of the three-axis components and then uses the root number as the denominator: when the X-axis component is a numerator, the angle of the X-component in the three axes can be calculated; when the Y-axis component is the numerator, the angle between the Y-component in the three axes can be calculated; when the Z-axis component is the numerator, the angle between the Z-component in the three axes can be calculated. After obtaining the angles between the three-dimensional coordinates and the X-axis, Y-axis, and Z-axis, the processor 21 can infer the posture of the human body by a known rule (or a preset rule) and the angle ranges falling between the X-axis, Y-axis, and Z-axis with the three-dimensional coordinates. However, those of ordinary skill in the art should understand that this is just an example, and the processor 21 can apply various existing pose algorithms to obtain the posture of the human body, which is not limited thereto.

In step 340, the processor 21 calculates the heart rate based on an ECG or the ECG signals, calculates an activity amount based on the three-axis data, and generates an activity intensity based on the activity amount, the user information, and the heart rate. In addition, the heart rate can selectively adopt the resting heart rate.

In one embodiment, the processor 21 calculates the heart rate according to the value in the ECG or the ECG signal.

In one embodiment, the electrocardiogram module 25 transmits the ECG signal to the processor 21. The processor 21 calculates the heart rate based on the heart electrical signal.

The following further describes the body-posture determination method 400, the activity amount calculation method 500, and the activity intensity generation method 600.

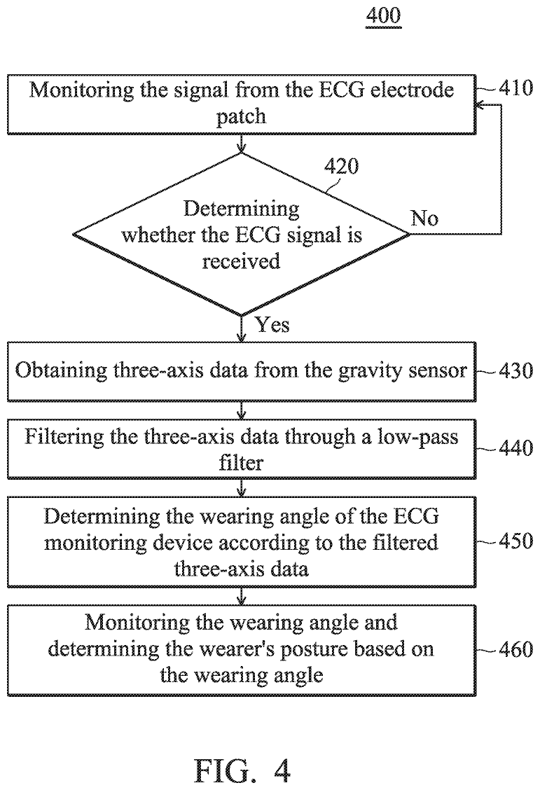

Please refer to FIG. 4, which is a flowchart of a body-posture determination method 400 in accordance with one embodiment of the present disclosure.

In step 410, the processor 21 monitors the signal from the ECG electrode patch 10.

In step 420, the processor 21 determines whether the ECG signal is received. If the processor 21 determines that the ECG signal is received, the step 420 is performed. If the processor 21 determines that the ECG signal is not received, for example, the ECG electrode patch 10 does not capture the change in the electrical potential of the skin surface, then the step returns to 410.

In step 430, the processor 21 obtains three-axis data from the gravity sensor 23.

In step 440, the processor 21 filters the three-axis data through a low-pass filter. The step can filter out noise and smooth the signal. In one embodiment, the low-pass filter can be implemented by known techniques.

In step 450, the processor 21 determines the wearing angle of the ECG monitoring device (for example, the ECG monitoring device 20) according to the filtered three-axis data.

In one embodiment, the processor 21 can continuously record the ECG signal for a certain period of time (e.g., 5 seconds) to determine that the ECG signal has been stabilized. In addition, the processor 21 then determines the wearing angle of the ECG monitoring device (for example, the ECG monitoring device 20 or 40) according to the filtered three-axis data. If the wearing angle represents a horizontal wearing angle, it is determined that the wearing method shown in FIG. 1A is adopted. If the wearing angle represents an oblique wearing angle (for example, an inclination of 45 degrees), it is determined that the wearing method shown in FIG. 2 is adopted.

In step 460, the processor 21 monitors the wearing angle and determines the wearer's posture based on the wearing angle.

In one embodiment, after knowing which the wearing methods is adopted, the processor 21 continuously monitors the wearing angle and determines the posture of the human body according to the change of the wearing angle.

In one embodiment, the change of the wearing angle refers to the relative change of the angle. For example, the processor 21 determines that the ECG monitoring device 20 is worn on the human body using the wearing method shown in FIG. 1A. The three-axis data indicates that the current human body is in an upright posture, thereby defining the initial wearing angle (e.g. defining the upright posture as 0 degrees). When the processor 21 detects that the wearing angle has changed (for example, from 0 degrees to 90 degrees), the processor 21 determines that the posture of the human body has changed. The processor 21 can be inferred from the change of the wearing angle that the human body may change from standing to lying on the side or lying down. The processor 21 can further obtain new three-axis data from the gravity sensor 23, and use the existing posture algorithm to more accurately determine that the posture of the human body has changed to lying on the side.

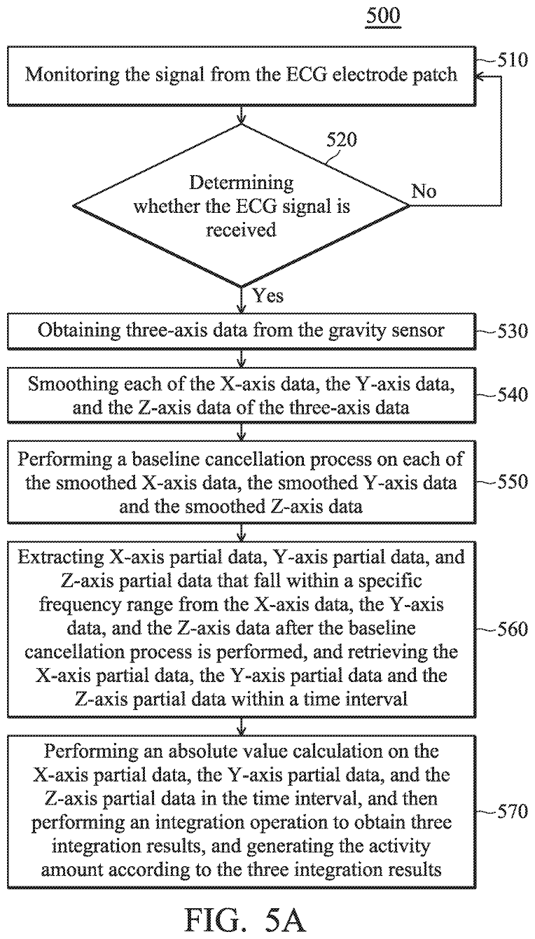

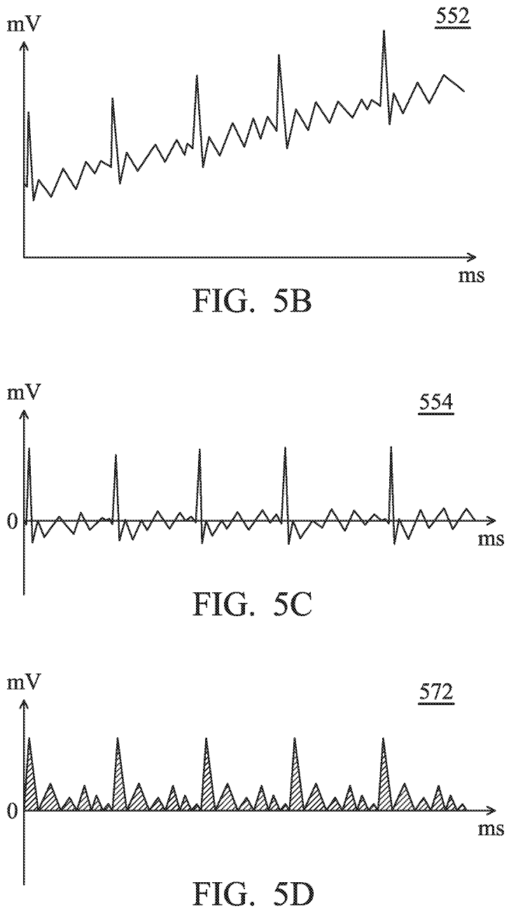

Please refer to FIGS. 5A-5D. FIG. 5A is a flowchart of an activity amount calculation method 500 in accordance with one embodiment of the present disclosure. FIG. 5B is a schematic diagram of X-axis data 552 of baseline drift according to an embodiment of the present invention. FIG. 5C is a schematic diagram of X-axis data 554 after baseline cancellation process according to an embodiment of the present invention. FIG. 5D is a schematic diagram of a line chart 572 for calculating the activity amount according to an embodiment of the present invention.

In FIG. 5A, the steps 510, 520, and 530 are the same as steps 410, 420, and 430 in FIG. 4, respectively, so details are not described here.

In step 540, the processor 21 smooths each of the X-axis data, the Y-axis data, and the Z-axis data of the three-axis data.

In one embodiment, the processor 21 respectively inputs the X-axis data, the Y-axis data, and the Z-axis data in the three-axis data to a moving average model for smoothing processing, thereby filtering noise.

In one embodiment, the X-axis data includes the X-axis component, the X-axis acceleration, and/or the X-axis tilt angle of the ECG monitoring device 20 continuously sensing by the gravity sensor 23 over a period of time. The Y-axis data includes the Y-axis component, the Y-axis acceleration, and/or the Y-axis tilt angle of the ECG monitoring device 20 continuously sensing by the gravity sensor 23 over a period of time. The Z-axis data includes the Z-axis component, the Z-axis acceleration, and/or the Z-axis tilt angle of the ECG monitoring device 20 continuously sensing by the gravity sensor 23 over a period of time.

In step 550, the processor 21 performs a baseline cancellation process on each of the smoothed X-axis data, the smoothed Y-axis data and the smoothed Z-axis data.

In one embodiment, the three-axis data received by the processor 21 may have a drifting state. As shown in FIG. 5B, the data points of the X-axis data 552 slowly drift to the upper right with the timing. Factors that cause the baseline drift, for example, the examinee overly nervous, when the limb muscles tremble due to cold, it is easy to affect the ECG signal to cause generating abnormal waveforms. Or, the examinee limbs or body movements cause the baseline resulting in unstable or even resulting in abnormal waveforms. By performing a baseline cancellation process on each of the smoothed X-axis data, the smoothed Y-axis data, and the smoothed Z-axis data, the X-axis data, Y-axis data, and Z-axis data can be maintained on a substantially horizontal line. Common baseline cancellation process methods such as automatic or manual search for the baseline, assigning a plurality of data points in the X-axis data, Y-axis data, and Z-axis data as the baseline, using interpolation or a nonlinear fitting function to find the best baseline. After finding the baseline, the processor 21 adjusts the baseline to a horizontal line, for example, rotating or translating the X-axis data, so as to move the baseline to a horizontal line. The adjusted X-axis data is, for example, the X-axis data 554 shown in FIG. 5C. The baseline cancellation process method is not limited thereto, and it can be implemented by a known algorithm.

The processor 21 performs baseline cancellation process on the X-axis data, Y-axis data, and Z-axis data in the same manner, thereby maintaining the X-axis data, Y-axis data, and Z-axis data in a horizontal line or within a range.

In step 560, the processor 21 uses a band-pass filter to extracts X-axis partial data, Y-axis partial data, and Z-axis partial data that fall within a specific frequency range through a band-pass filter from the X-axis data, the Y-axis data, and the Z-axis data after the baseline cancellation process is performed, and the processor 21 retrieves the X-axis partial data, the Y-axis partial data and the Z-axis partial data within a time interval.

In one embodiment, the bandwidth is the frequency range in which the gravity sensor 23 operates. For example, the X-axis data, Y-axis data, and Z-axis data that have undergone baseline cancellation process may contain data with 0 to 120 Hz bandwidth range. In order to reduce the amount of data to be processed or to process data with a specific bandwidth, the data in the 20 to 80 Hz bandwidth range can be retrieved through the band-pass filter.

In one embodiment, the processor 21 retrieves the X-axis partial data, the Y-axis partial data, and the Z-axis partial data within a time interval (e.g., within 2-7 seconds).

In step 570, the processor 21 performs an absolute value calculation on the X-axis partial data, the Y-axis partial data, and the Z-axis partial data in the time interval, and then performs an integration operation to obtain three integration results, and generates the activity amount according to the three integration results.

In one embodiment, the processor 21 can obtain the line graph 572 of the FIG. 5D graph after the processor 21 performs an absolute value calculation on the X-axis partial data. It can be seen from the line graph 572 that all values are positive values, and the processor 21 calculates the gray block area (the processor 21 performs an integration operation on the line graph 572), and the integration result corresponding to the X-axis partial data can be obtained. The processor 21 performs the same processing on the X-axis partial data, the Y-axis partial data and the Z-axis partial data to obtain three integration results.

In one embodiment, the processor 21 may add up, multiply, and multiply each of the three integrals by a weight, and then add up or perform other operations, and regard the obtained value as an activity amount. For example, the integral of the X-axis partial data is 100, the integral of the Y-axis partial data is 200, and the integral of the Z-axis partial data is 300. The processor 21 adds up the three integrals to obtain 600, and regards the activity amount as 600. In one embodiment, the activity amount may be a numerical value to quantify the degree of activity of the examinee each time when the measurement is performed (e.g., within 5 minutes).

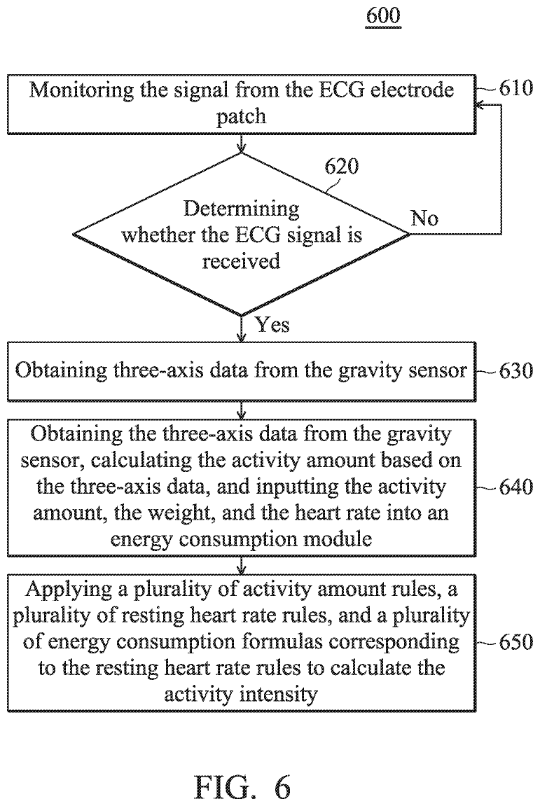

Please refer to FIG. 6, FIG. 6 is a flowchart of an activity intensity generation method 600 in accordance with one embodiment of the present disclosure. In FIG. 6, the steps 610, 620, and 630 are the same as steps 410, 420, and 430 in FIG. 4, respectively, so details are not described here.

In step 640, the processor 21 obtains three-axis data from the gravity sensor 23, calculates the activity amount based on the three-axis data, and inputs the activity amount, the weight, and the heart rate into an energy consumption module. In addition, the heart rate can selectively adopt the resting heart rate.

In one embodiment, the energy consumption module is used to execute an algorithm, which is used to calculate the activity intensity.

In one embodiment, the energy consumption module can be implemented by a hardware circuit, firmware or software.

In one embodiment, the energy consumption module is a program that can be executed by the processor 21.

In one embodiment, the energy consumption module is stored in the storage device 27. The processor 21 inputs the activity amount, the weight and the heart rate to the energy consumption module (which can be an algorithm), and the energy consumption module outputs the activity intensity. In addition, the heart rate can selectively adopt the resting heart rate.

In one embodiment, the energy consumption module is composed of a hardware circuit, which is electrically coupled to the processor 21. The processor 21 inputs the activity amount, the weight and the heart rate to the energy consumption module for calculation. The energy consumption module then outputs the activity intensity. In addition, the heart rate can selectively adopt the resting heart rate.

In step 650, the energy consumption module applies a plurality of activity amount rules, a plurality of resting heart rate rules, and a plurality of energy consumption formulas corresponding to the resting heart rate rules to calculate the activity intensity.

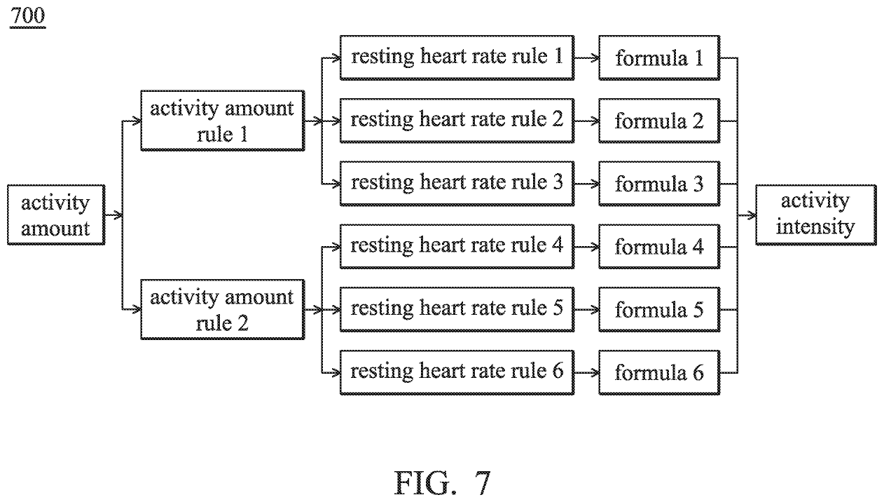

Please refer to FIG. 7, FIG. 7 is a schematic diagram illustrating an activity intensity generation method 700 according to an embodiment of the present invention. The activity intensity generation method 700 can be executed by the energy consumption module.

After the processor 21 inputs the activity amount (activity amount can be calculated by activity amount calculation method 500), the weight (the weight can be inputted by the user), and the heart rate (the heart rate can be calculated by the processor 21 according to the value in the ECG or the ECG signal) to the energy consumption module, such information may be temporarily stored in the storage device 27 or the storage space that the energy consumption module itself has or corresponds to.

The energy consumption module firstly takes out the activity amount, for example, the activity amount is 10. Then, it is determined whether the activity amount meets the activity amount rule 1 (for example, greater than 152) or the activity amount rule 2 (for example, less than or equal to 152). In this example, the activity amount of 10 belongs to a range of less than or equal to 152, which complies with activity amount rule 2. When the active amount complies with the active amount rule 2 are shown in FIG. 7, the energy consumption module further determines that the heart rate conforms to the resting heart rate rule 4, the resting heart rate rule 5 or the resting heart rate rule 6.

Next, the energy consumption module takes out the value of the heart rate, for example, 55 (the number of heart beats per minute is 55), the energy consumption module compares the value of the heart rate with the resting heart rate rule 4 (for example, less than 40), and the resting heart rate rule 5 (for example, greater than or equal to 40 and less than or equal to 65) and resting heart rate rule 6 (for example, greater than 65). If the heart rate meets the resting heart rate rule 4, then execute formula 4. If the heart rate meets the resting heart rate rule 5, then execute formula 5. If the heart rate meets the resting heart rate rule 6, execute formula 6. In this example, the heart rate is 55, which meets the resting heart rate rule 5, so formula 5 is executed.

In one embodiment, formula 5 is, for example, the basal metabolic rate (BMR) multiplied by 1.2 to obtain the value as the activity intensity. Assuming that the basal metabolic rate is 1100, and 1320 obtained by multiplying 1100 by 1.2 is taken as the activity intensity (the unit of the activity intensity may be kilograms multiplied by minutes of calories (cal./(kg*min)). In addition, the basal metabolic rate can be calculated by a known function (such as the Mifflin St Jeor Equation function). Finally, the energy consumption module outputs the activity intensity.

In another example, the energy consumption module firstly takes out the activity amount, for example, the activity amount is 180. Then, it is determined whether the activity amount meets the activity amount rule 1 (for example, greater than 152) or the activity amount rule 2 (for example, less than or equal to 152). In this example, the activity amount of 180 belongs to a range of greater than 152, which complies with activity amount rule 1. When the active amount complies with the active amount rule 1 are shown in FIG. 7, the energy consumption module further determines that the heart rate conforms to the resting heart rate rule 1, the resting heart rate rule 2 or the resting heart rate rule 3.

Next, the energy consumption module takes out the value of the heart rate, for example, 80 (the number of heart beats per minute is 80), the energy consumption module compares the value of the heart rate with the resting heart rate rule 1 (for example, less than 40), and the resting heart rate rule 2 (for example, greater than or equal to 40 and less than or equal to 65) and resting heart rate rule 3 (for example, greater than 65). If the heart rate meets the resting heart rate rule 1, then execute formula 1. If the heart rate meets the resting heart rate rule 2, then execute formula 2. If the heart rate meets the resting heart rate rule 3, execute formula 3. In this example, the heart rate is 80, which meets the resting heart rate rule 3, so formula 3 is executed.

In one embodiment, formula 3 is, for example, the basal metabolic rate multiplied by 1.95 to obtain the value as the activity intensity. Assuming that the basal metabolic rate is 1200, and 2340 obtained by multiplying 1200 by 1.95 is taken as the activity intensity (the unit of the activity intensity may be kilograms multiplied by minutes of calories (cal./(kg*min)). In addition, the basal metabolic rate can be calculated by a known function (such as the Mifflin St Jeor Equation function). Finally, the energy consumption module outputs the activity intensity.

In an embodiment, formula 1 to formula 6 can be the same or different formulas. For example, formula 1 can be multiplied by 1.55 to obtain the activity intensity. Formula 2 can be multiplied by 1.725 to obtain the activity intensity. Formula 3 can be obtained by multiplying the basal metabolic rate by 1.95 to obtain the activity intensity. Formula 4 is, for example, multiplying metabolic equivalent of tasks (METs, unit: kilometers/hour (km/hr)) by 1.2 and multiplying by body weight (kg) to obtain the activity intensity. Formula 5 is, for example, multiplying the basal metabolic rate by 1.2 to obtain the activity intensity. Formula 6 is, for example, multiplying the METs (km/hr) by 1.5 and then by the body weight (kg) to obtain the activity intensity.

METs can be understood as the energy consumption level in a specific activity state relative to the resting metabolic state (quiet sitting and resting). The value range of the METs is from 0.9 (e.g., when sleeping) to 23 (e.g., when running at 22.5 km/h). It means that the body's energy consumption level may reach 0.9 times of the resting metabolic state when sleeping, and the body's energy consumption level may reach 23 times of the metabolic state when running at high speed. The value of METs can be found by looking up the table based on the posture of the human body. For example, the posture of the human body is lying down, which means that the human may be sleeping. The energy consumption module looks up a comparison table to obtain the values of METs corresponding to sleeping activity. This comparison table is known information, which contains the values of METs corresponding to multiple exercises. The contents of the above formulas 1 to 6 are only examples, it is not limited thereto, and the contents of each formula 1 to formula 6 can be adjusted according to actual implementation.

In one embodiment, since the processor 21 can receive the ECG signal and at same time the three-axis data can be obtained by the gravity sensor 23, the processor 21 calculates the posture of the human body, the activity amount and the activity intensity at the current time, and stores such information in the storage device 27, and/or is transmitted to the electronic device 30 by the transmission device 29.

When the ECG module 25 receives the ECG signal, the processor 21 and/or the electronic device 30 can immediately note the patient's posture, the activity amount and the activity intensity corresponding to each time point on the ECG. In addition, after obtaining the ECG, the processor 21 and/or the electronic device 30 can also label the posture, activity amount, and the activity intensity of the human body corresponding to each time point on the ECG in an offline state. In this way, the user can click on a certain time point of the ECG on the electronic device 30 or the display interface of the physiological signal processing system to know the wearer's posture, the activity amount and the activity intensity at a certain time point.

The physiological signal processing method and physiological signal processing system shown in the present invention can output the posture, the activity amount and the activity intensity of the human body in combination with the waveform of the ECG, in order to allow the user to know the wearer's posture, the activity amount and the activity intensity corresponding to each time point of the ECG. Labeling the posture, the activity amount and the activity intensity at each time point of the ECG can assist medical personnel in interpreting ECGs more accurately, and preventing the ECG from becoming distorted due to the patient's posture or activity during an electrocardiogram, making interpretation difficult for medical personnel.

What is claimed is:

1. A physiological signal processing system, comprising:
an electrocardiogram (ECG) monitoring device, comprising:
a processor, configured to receive a plurality of ECG signals and user information;
an ECG module, configured to capture the ECG signals and transmit the ECG signals to the processor; and
a gravity sensor (g-sensor), configured to detect a displacement state of the ECG monitoring device to obtain three-axis data;
wherein the processor calculates a heart rate based on an ECG or the ECG signals, calculates an activity amount based on the three-axis data, and generates an activity intensity based on the activity amount, the user information, and the heart rate.

2. The physiological signal processing system of claim 1, further comprising:
an ECG electrode patch, configured to obtain the ECG signals;
wherein the ECG electrode patch is pasted under the right shoulder blade of a human body, and the ECG signals are transmitted by wire to the ECG monitoring device located below the left chest;
wherein the ECG monitoring device is worn on the human body at a horizontal wearing angle.

3. The physiological signal processing system of claim 1, further comprising:
an ECG electrode patch, configured to obtain the ECG signals;
wherein the ECG electrode patch is affixed to the ECG monitoring device, and the ECG electrode patch is obliquely attached to the left chest of the human body at an oblique wearing angle;
wherein the ECG monitoring device is worn on the human body at an oblique wearing angle.

4. The physiological signal processing system of claim 1, wherein the processor is further used to determine whether the ECG signals are received;
if the processor determines that the ECG signals are received, the processor obtains the triaxial data from the gravity sensor, inputs the three-axis data into a low-pass filter for filtering, determines a wearing angle of the ECG monitoring device based on the filtered three-axis data, monitors the wearing angle, and determines a human posture based on the wearing angle.

5. The physiological signal processing system of claim 1, wherein the processor is further used to determine whether the ECG signals are received;
if the processor determines that the ECG signals are received, the three-axis data is obtained by the gravity sensor, and the processor respectively smoothes the X-axis data, the Y-axis data and the Z-axis data in the three-axis data, the processor respectively performs a baseline cancellation process on each of the smoothed X-axis data, the smoothed Y-axis data, and the smoothed Z-axis data, the processor extracts X-axis partial data, Y-axis partial data, and Z-axis partial data that fall within a specific frequency range through a band-pass filter from the X-axis data, the Y-axis data, and the Z-axis data after the baseline cancellation process is performed, and the processor retrieves the X-axis partial data, the Y-axis partial data and the Z-axis partial data within a time interval, the processor performs an absolute value calculation on the X-axis partial data, the Y-axis partial data, and the Z-axis partial data in the time interval, and then performs an integration operation to obtain three integration results, and the processor generates the activity amount according to the three integration results.

6. The physiological signal processing system of claim 5, wherein the user information includes a weight, and the processor is further used to determine whether the ECG signals are received, and if the processor determines that the ECG signals are received, the three-axis data is obtained from the gravity sensor, the activity amount is calculated according to the three-axis data, and the activity amount, the weight, and the heart rate are input into an energy consumption module, wherein the energy consumption module applies a plurality of activity amount rules, a plurality of resting heart rate rules, and a plurality of energy consumption formulas corresponding to the resting heart rate rules to calculate the activity intensity.

7. A physiological signal processing method, comprising:
receiving a plurality of ECG signals and user information;
capturing the ECG signals;
detecting the displacement state of the ECG monitoring device to obtain three-axis data;
calculating the heart rate based on an ECG or the ECG signals, calculating an activity amount based on the three-axis data; and
generating an activity intensity based on the activity amount, the user information, and the heart rate.

8. The physiological signal processing method of claim 7, further comprising:
determining whether the ECG signals are received; wherein if the ECG signals are received, then the following steps are performed:
obtaining the triaxial data from the gravity sensor, filtering the three-axis data;
determining the wearing angle of the ECG monitoring device based on the filtered three-axis data; and
monitoring the wearing angle, and determining a human posture based on the wearing angle.

9. The physiological signal processing method of claim 7, further comprising:
determining whether the ECG signals are received; wherein if the ECG signals are received, then the following steps are performed:
obtaining the three-axis data, and respectively smoothing the X-axis data, the Y-axis data and the Z-axis data in the three-axis data;
performing a baseline cancellation process on each of the smoothed X-axis data, the smoothed Y-axis data, and the smoothed Z-axis data, respectively;
extracting X-axis partial data, Y-axis partial data, and Z-axis partial data that fall within a specific frequency range from the X-axis data, the Y-axis data, and the Z-axis data after the baseline cancellation process is performed; and
retrieving the X-axis partial data, the Y-axis partial data and the Z-axis partial data within a time interval, performing an absolute value calculation on the X-axis partial data, the Y-axis partial data, and the Z-axis partial data in the time interval, and then performing an integration operation to obtain three integration results, and generating an activity amount according to the three integration results.

10. The physiological signal processing method of claim 9, wherein the user information includes a weight, the physiological signal processing method further comprising:
determining whether the ECG signals are received; wherein if the ECG signals are received, then the following steps are performed:
obtaining the three-axis data, and calculating the activity amount according to the three-axis data;
inputting the activity amount, the weight, and the heart rate into an energy consumption module, wherein the energy consumption module applies a plurality of activity amount rules, a plurality of resting heart rate rules, and a plurality of energy consumption formulas corresponding to the resting heart rate rules to calculate the activity intensity.
